(19) United States
(12) Patent Application Publication
(10) Pub. No.: US 2024/0415743 A1
(43) Pub. Date: Dec. 19, 2024

(54) SHAMPOO IN SOLID FORM TO BE RECONSTITUTED IN AN AQUEOUS ENVIRONMENT

(71) Applicant: LES SOLUTIONS ZERO DECHET FILLGOOD INC., Saint-Augustin-de-Desmaures (CA)

(72) Inventor: Sophie FORTIN, Quebec (CA)

(21) Appl. No.: 18/705,117

(22) PCT Filed: Oct. 24, 2022
(86) PCT No.: PCT/CA2022/051567

§ 371 Date: Apr. 26, 2024

Related U.S. Application Data
(60) Provisional application No. 63/271,767, filed on Oct. 26, 2021.
(60) Provisional application No. 63/338,516, filed on May 5, 2022.

(51) Int. Cl. A61K 8/02; A61K 8/36; A61K 8/46; A61K 8/73; A61K 8/9717; A61Q 5/02

(52) U.S. Cl. CPC A61K 8/0216; A61K 8/36; A61K 8/466; A61K 8/73; A61K 8/9717; A61Q 5/02; A61K 2800/48; A61K 2800/524

(57) ABSTRACT

The present technology generally relates to a shampoo in solid form including: a system of surfactants, a system of thickening agents, at least one conditioning agent, a system of preservation agents. The shampoo being reconstitutable to be used in liquid form. This technology also relates to a method of preparing shampoo in liquid form.

### FIELD OF TECHNOLOGY

In general terms, this technology relates to a formulation of shampoo in solid form to be reconstituted in an aqueous environment.

### BACKGROUND

Shampoos available on the market are mainly made up of water. It can even represent up to 80% of the product by mass, which is significant. Therefore, the consumer is largely paying for water, transport of water and a bottle, usually made of plastic. Given that the majority of the population has access to high quality water at home, there is no need to have it travel such long distances. Consequently, a concentrated shampoo in solid form allows for a 95% reduction of CO₂ emissions connected to the transportation of water in traditional products. A tablet of shampoo or a shampoo powder represents a volume about 200 times smaller and a weight 300 times lower than conventional shampoo, therefore a proportionate saving on transport.

So, shampoos currently available on the market have disadvantages that would benefit from improvement.

### SUMMARY

One aspect of the present technology relates to a shampoo in solid form to be reconstituted in water for medium-term use. When it dissolves in water, shampoo forms a liquid of a consistency similar to conventional shampoos. The liquid shampoo that is formed is comparable to traditional shampoo in so far as it has features that are typically found in traditional shampoo.

Another aspect of this technology relates to a shampoo in solid form comprising: a system of surfactants, a system of thickening agents, at least one conditioning agent, a system of preservation agents, wherein the shampoo is to be reconstituted to a liquid form.

Another aspect of this technology relates to a method for reconstituting the shampoo in solid form to a liquid form. The method comprises solubilizing the shampoo in solid form in a quantity of aqueous solvent in order to create a solubilized shampoo; and shaking the solubilized shampoo.

### DETAILED DESCRIPTION

The description of well-known ingredients, manufacturing tools and processing techniques has been omitted in order to provide a clearer illustration of the technology in detail. However, it should be understood that the detailed description and specific examples, whilst indicating the embodiment of the technology, are given for illustrative purposes only, and are not restrictive. Various substitutions, modifications, additions and/or adjustments to the spirit and scope of the underlying inventive concepts will be recalled by those familiar with the technological field.

The terminology used here aims to describe only the particular embodiments and is not intended to be restrictive where the technology is concerned. As used here, the singular forms “one”, “the” are also intended to include the plural form, unless the context clearly indicates otherwise. Furthermore, it is well understood that the terms “comprise” (and all forms of “to comprise” such as “comprises” and “comprising”), “to have” (as well as all forms of “to have” such as “had” and “have”), “to include” (and all forms of inclusions such as “includes” and “including” and “contains” (as well as all forms of “to contain” such as “should contain” and “contain”) are open-ended crossover verbs.

According to some aspects, this technology relates to a shampoo in solid form to be reconstituted in an aqueous environment, such as for example, water. In some embodiments, the shampoo in solid form is a powder. In other embodiments, the shampoo in solid form is a tablet.

According to some aspects, shampoo from this technology is for medium-term use. For example, medium-term use means use within a period of around one (1) month to around six (6) months.

Once fully dissolved in an aqueous environment and having reached a consistency comparable to that of honey, the shampoo is of a consistency similar to conventional shampoos. Once reconstituted, the resulting liquid shampoo is no different in texture, scent or lather, thus creating a gentle, revitalizing shampoo from a tablet or a powder and a prescribed amount of water.

Currently, the market offers a wide range of liquid shampoos. However, there are no shampoos in solid form (e.g., in tablet or powder form) to be reconstituted in liquid form for medium-term use and offering the same capillary properties.

According to some aspects, this technology relates to shampoo in solid form to be reconstituted for medium-term use, which, on contact with a pre-determined quantity of aqueous solvent, becomes hydrated and thickens to form a traditional shampoo. In some aspects of the present technology, the shampoo in solid form is in a powder form. In other aspects, the shampoo in solid form is a tablet. In some aspects of this technology, the ratio of tablet or powder in the aqueous solvent (e.g., water) is around 50 g of shampoo for around 350 g of water, therefore around 350 ml of water. The final total volume of shampoo made up is around 400 mL. The reconstituted shampoo has detergent and lathering properties similar to those of conventional shampoos as well as benefits to the hair (hydration, anti-dandruff, de-tangling, etc.) due to the addition of specific conditioning agents, in some embodiments.

According to one or more embodiments, at least some of the ingredients used in preparing shampoo in solid form from this technology must have a high level of solubility in water, meaning that on contact with the required quantity of water, at least some of the ingredients will be soluble. Alternatively, one or more ingredients that would not be completely soluble could require the addition of a solubilizing or dispersing agent, in some embodiments. Depending on the way the tablet or powder shampoo is made up, the gelling agents used must have a high level of solubility in cold water and are easily dispersed.

The term “reconstitution” means that the shampoo in solid form is dissolved in the quantity of aqueous solvent before use.

The term “gentle” refers to the shampoo in solid form being formulated with surfactants that respect the hair and the scalp. Therefore, it is about avoiding sulphate compounds, for example, but not exclusively, ammonium laureth sulphate, ammonium lauryl sulphate, sodium laureth sulphate and sodium lauryl sulphate.

The expression “revitalizing effect” means that after using the reconstituted shampoo, the hair has greater manageability and once dry, the hair is soft and silky to the touch.

In some aspects, the shampoo in solid form according to this technology includes: at least one surfactant, at least one thickening agent, at least one conditioning agent, at least one preservation agent, and optionally, one or more additives.

Shampoo in solid form from this technology, when dissolved in an aqueous solvent, forms a liquid of a similar consistency to conventional shampoos. Once, reconstituted, the resulting liquid shampoo is no different in texture, scent and lather, thus creating a gentle, revitalizing shampoo from a tablet or powder and a prescribed quantity of water. To be gentle, the shampoo must have a pH level suitable for that of the hair, that is to say slightly acidic, in order to smooth the hair cuticles and not de-stabilize the capillary environment. This means that the shampoo has a pH between 4.5 and 7, ideally between 4.5 and 5.5, but without being restricted to these values. The revitalizing effect means that the reconstituted shampoo leaves hair moisturized, shining and soft after use.

According to some embodiments, the at least one surfactant included in the shampoo in solid form of the present technology is a primary surfactant. Examples of primary surfactants useful in practicing this technology include all solid, non-sulphate and natural surfactants or those derived from natural sources. They may include, but are not restricted to, sodium lauroyl sarcosinate, sodium lauryl sulphoacetate, sodium olivate, sodium lauroyl glutamate, sodium cocoate, glyceryl oleate, sodium cocoyl glutamate, lauryl glucoside, coco-glucoside, lauroyl arginine, sodium cocoyl glycinate or sodium cocoyl isethionate. In addition to their detergent properties, these surfactants may also act as lathering agents, increasing the lather, stabilizing the lather and act as emulsifiers and dispersing, wetting or solubilizing agents. Some may also have hair conditioning properties and skin maintenance agents. In some embodiments, the primary surfactant may be sodium lauryl sulphoacetate, which is commercially available in the form of a white powder and from several sources. The content of primary surfactant, such as sodium lauryl sulphoacetate, to obtain the cleaning effect and the desired lather may be between about 10% and about 60% by mass of shampoo. In some embodiments, the content of primary surfactant may be about 15%, about 25%, about 30%, about 40%, about 50% or about 60% per total mass of the shampoo.

In some embodiments, the shampoo in solid form may be in the form of loose powder or be compressed into tablet form. In the latter aspect, this technology therefore also relates to shampoo tablets made up of shampoo powder, from the current technology that has been compressed. In some embodiments, the content of primary surfactant may be about 15%, about 25%, about 30%, about 40%, about 50% or about 60% per total mass of the powder.

In some embodiments, the at least one surfactant included in the shampoo of the present technology is a secondary surfactant. Examples of secondary surfactants, that may be useful for this technology include, but are not restricted to, all the surfactants listed previously. In some embodiments, the secondary surfactant may be sodium cocoyl glutamate, which is commercially available in the form of a white powder and is available from several sources. The content of secondary surfactant, such as sodium cocoyl glutamate, may be between about 5% to about 35% of total mass of powder. In some embodiments, the content of secondary surfactant may be about 10%, about 15%, about 20%, about 25%, about 30% or about 35% of total mass of the shampoo.

In some embodiments, a contour surfactant may be used combined with the primary and secondary surfactants. The contour surfactant may include, but is not restricted to, all the surfactants listed previously. In some embodiments, the contour surfactant may be sodium cocoyl glycinate, which is commercially available in the form of a white powder, and is available from several sources. The content of contour surfactant, such as sodium cocoyl glycinate, may be between about 0.1% and about 10% per total mass of powder. In some embodiments, the content of secondary surfactant may be about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, or about 9% of total mass of the shampoo.

Thickening agents can be useful in the practice of this technology and include thickeners that must have a high level of solubility in cold water and are easily dispersed with minimal agitation, to be compatible with the method of reconstituting the shampoo in the form of tablets or powder and in order to form a liquid of the desired consistency. They may include, but are not restricted to, dehydrated xanthan gum, xanthan gum, cellulose gum, sodium alginate or carrageenan. In some embodiments, the thickening agent may be xanthan gum, which is commercially available in loose powder form in a white to cream colour and from several sources. The content of thickening agent, such as xanthan gum, to obtain the desired viscosity may be between about 1% and about 30% of total mass of shampoo. In some embodiments, the content of thickening agent may be about 5%, about 10%, about 15% or about 20% of total mass of shampoo. In some embodiments, the thickening agent may be carrageenan, which is commercially available in the form of loose powder in a white to yellowish color, and from several sources. The content of thickening agent, such as carrageenan, to obtain the desired viscosity may be between about 5% and about 20% of total mass of the shampoo. In some embodiments, the content of thickening agent may be about 5%, about 10%, about 15% or about 20% of total mass of the shampoo.

In some embodiments, salts may be added in order to help modulate the consistency of the reconstituted tablet or powder shampoo, for example, but not restricted to, sodium chloride. Solubility in cold water and ability to easily disperse with minimal agitation, compatible with the recommended method of reconstitution, means that a texture equivalent to that of conventional shampoos can be obtained. It is understood that one or more thickening agents may be used in order to obtain the desired consistency, comparable in quality to that of shampoos from indirect competitors.

Conditioning agents may be present in the shampoo of the present technology in order that hair can benefit from cosmetic qualities and advantages as a result of the reconstituted shampoo. It includes solid conditioning agents and those which are easily dissolved in cold water to be compatible with the method of reconstitution of shampoo in solid form in the prescribed amount of water. They may include, but are not restricted to, sarcosine, protein hydrolysates, plant powders, starches, vegetable oils or vegetable milks. In some embodiments, the conditioning agent may include rice starch, which is commercially available in the form of a white powder, and from several sources. The content of conditioning agent, such as rice starch, required to obtain the desired effects on the hair, may be between about 0.1% and about 10% per total mass of the shampoo. In some embodiments, the content of conditioning agent may be about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, or about 9% per total mass of the shampoo. It is understood that one or more conditioning agents may be used in order to obtain the desired effects on the hair.

Preservation agents may be present in the shampoo according to this technology in order to protect the reconstituted shampoo from mold, yeasts and bacteria. Examples of preservation agents that are useful in this technology include solid preservation agents that are easily dissolved in cold water to be compatible with the method of reconstituting the shampoo in the prescribed amount of water. They may include, but are not restricted to, grapefruit seed extract, gluconolactone, sodium benzoate, naticide, leucidal or potassium sorbate. In some embodiments, the preservation agent may be gluconolactone, which is commercially available in the form of white crystalline powder, and from several sources. The content of the preservation agent, such as gluconolactone, to obtain the desired factor of protection may be between about 0.1% and about 10% per total mass of shampoo. In some embodiments, the content of preservation agents may be about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, or about 9% per total mass of the shampoo. In some embodiments, the preservation agent may be sodium benzoate, which is commercially available in the form of a white powder, and from several sources. The content of the preservation agent, such as sodium benzoate, to obtain the desired protection factor may be between about 0.1% and about 5% per total mass of the shampoo. In some embodiments, the content of preservation agent may be about 0.5%, about 1%, about 1.5%, about 2%, about 2.5%, about 3%, about 3.5%, about 4%, about 4.5%, or about 5% per total mass of the shampoo. It is understood that one or more conditioning agents may be used.

Shampoo in solid form, according to some embodiments from this technology, may further comprise one or more additional additives in order to optimize its formulation. The additives may include, for example, some chelating agents, pH adjusters, fragrances or coloring agents, without being restricted to these. When the latter are present in the formula of the shampoo in solid form to be reconstituted with the aqueous solvent for medium-term use, they may be present at levels of between about 0.01% and about 7% per total mass of the shampoo. According to some embodiments, the content by mass in additives may include about 1%, about 2%, about 3%, about 4% about 5% or even about 7% per total mass of the shampoo. In some embodiments, at least one chelating agent can be used, which could include, for example, tetrasodium glutamate diacetate, sodium gluconate or sodium citrate. In some production methods, at least one pH adjuster may be used, which may include, for example, citric acid, sodium sesquicarbonate or cream of tartar. In addition, fragrances may be used, which could include all fragrances derived from natural sources, including, but not restricted to, aromatic oils, essential oils or extracts of supercritical CO₂. In some embodiments, one or more colorings may be used, comprising but not restricted to, natural powder colorings, liquid colorings or vegetable or fruit extracts.

In some embodiments, additional additives may not be supplied in a solid form like the other ingredients so that the final shampoo in the form of tablets or powder is dry after they have been added. The ingredients in powder form, therefore dry, must be able to absorb their humidity or oily nature in order to ensure that the powder finally obtained is dry or substantially dry. Otherwise, the solvent of the additives must evaporate during production, during mixing or during a rest period. As used in this description, substantially dry may include less than about 5%, or less than about 4% or less than about 3% or less than about 2%, or less than about 1% humidity.

In some embodiments, absorbing agents may be present in the shampoo in solid form in order to assist incorporation of fragrances. Examples of absorbing agents that are useful in this technology include solid absorbing agents, easily dissolved in cold water to be compatible with the reconstitution method of shampoo in powder form in the prescribed amount of water. They may include, but are not restricted to cyclodextrin, tapioca starch, cornstarch or maltodextrin. In some embodiments, the absorbing agent may be maltodextrin, which is commercially available in the form of white crystal powder, and from several sources. The content of absorbing agent, such as maltodextrine, to facilitate incorporation of the fragrance may be between about 0.1% and about 5% per total mass of the shampoo. In some embodiments, the content of absorbing agent may be about 0.5%, about 1%, about 1.5%, about 2%, about 2.5%, about 3%, about 3.5%, about 4%, about 4.5%, or about 5% of total mass of the shampoo.

The shampoo in solid form according to this technology may be prepared, in general, by combining loose powder in an appropriate container in order to create a powdery mixture of the aforementioned ingredients. As it is used, the powder may refer to any solid ingredient in bulk, fine or untreated, comprising but not limited to, dust, granules or crystalline powder. The term loose powder refers to the fluidity of the powder, more specifically to its ability to flow in the preparation stages required to mix the shampoo in powder form.

Powders that do not flow freely may be added to the mix. Powders that do not flow freely may include, but are not restricted to, essential or aromatic oils, vegetable oil powders, powdered fragrances and powders that are dispersible but not soluble, and which may have emollient or exfoliating properties. The final mix in the form of a homogeneous powder may be obtained by any appropriate method. In some embodiments, an electric grinder may be used to prepare the powders, and also to mix the ingredients together. A ribbon blender could also be used to mix the powders and the obtained mixture would then be passed through a sieve.

In some embodiments, the shampoo in powder form may be compressed to form a soluble tablet to be reconstituted in an aqueous solvent. Where the powder is compressed to form a tablet, dissolving agents may be present in the shampoo in powder form in order to facilitate dissolution of the tablet in the aforementioned aqueous solvent. Examples of dissolving agents that are useful in this technology include dissolving agents that assist the dissolution of the solids and have a good level of solubility in cold water to be compatible with the method of reconstituting the shampoo in tablet form in the prescribed amount of water. They may include, but are not restricted to citric acid, sodium bicarbonate, erythritol or microcrystalline cellulose. In some embodiments, the dissolving agent may be citric acid, which is commercially available in the form of a white crystalline powder, and from several sources. In some embodiments, citric acid may be used together with sodium bicarbonate, which is commercially available in the form of a white crystalline powder, and from several sources. The total content of dissolving agents to facilitate dissolution of compressed powder in tablet form may be between about 10% and about 60% by mass of powder. In some embodiments, the total content of dissolving agents may be about 15%, about 25%, about 30%, about 40%, about 50%, or about 60% per total mass of powder.

Once the ingredients are mixed, the shampoo in solid form is completely or nearly completely dry. Once solubilized and rehydrated in the prescribed amount of water, the reconstituted shampoo may be used like a conventional shampoo, in so far as it has a similar consistency. The water used for dissolution and rehydration does not need to be at a specific temperature, in so far as the reconstitution will work with cold, tepid, warm or hot water. A mass of around 50 grams of tablet or powder must be added to a quantity of 350 ml of water, in the bottle provided, to obtain the correct amount of each of the ingredients. A gentle mix, by inverting the bottle wets the ingredients, to facilitate the dissolution and dispersion, without creating too much foam. In some embodiments, it is recommended that the product is not shaken too much to avoid compromising the final texture. A waiting time of up to about 6 (six) hours may be needed to allow for complete dissolution and development of optimal viscosity. Once the waiting time has passed, the reconstituted shampoo in powder form may be used like a conventional shampoo.

In some embodiments, shampoo in solid form is exempt from certain ingredients that are deemed to have a negative impact on the health of the planet and human beings. For example, the shampoo in solid form of the present technology to be reconstituted in water may be exempt from parabens, synthetic fragrances, artificial colorings, sulphates, silicones, ingredients derived or originating from animals, mineral oil, of PEGs or ingredients derived from the petrochemical industry.

According to some embodiments, the shampoo in solid form of the present technology may comprise a majority of ingredients of natural origin or derived from natural or synthetic sources, but identical to those found in nature, and biodegradable.

The shampoo in solid form of the present technology, once reconstituted in the prescribed amount of water, may be used like a traditional shampoo. The resulting liquid shampoo may be applied to the user's hair for a period of time varying between about 30 seconds and 3 minutes. The period of time may include, for example, between around 20 seconds and 5 minutes, and about 1 minute and 2 minutes. In some embodiments, additional water may be added to the shampoo by adding water to the hair in order to control the density of the lather during use. The shampoo is then rinsed from the user's hair in the same way as conventional shampoo.

1. A shampoo in solid form, the shampoo comprising:
at least one surfactant;
at least one thickening agent;
at least one conditioning agent; and
at least one system of preservation agents;
the shampoo being reconstitutable in an aqueous environment to create a shampoo in a liquid form.

2. The shampoo in solid form according to claim 1, wherein the at least one surfactant is a primary surfactant.

3. The shampoo in solid form according to claim 2, wherein the primary surfactant is present in the shampoo in an amount of between about 10% and about 60% per total mass of the shampoo.

4. The shampoo in solid form according to claim 1, wherein the at least one surfactant is a secondary surfactant.

5. The shampoo in solid form according to claim 4, wherein the secondary surfactant is present in the shampoo in an amount of between about 5% and about 35% per total mass of the shampoo.

6. The shampoo in solid form according to claim 1, wherein the at least one surfactant is a contour surfactant.

7. The shampoo in solid form according to claim 6, wherein the contour surfactant is present in the shampoo in an amount of between about 0.1% and about 10% per total mass of the shampoo.

8. The shampoo in solid form according to claim 1, wherein the at least one surfactant is selected from: sodium lauroyl sarcosinate, sodium lauryl sulfoacetate, sodium olivate, sodium lauroyl glutamate, sodium cocoate, glyceryl oleate, sodium cocoyl glutamate, lauryl glucoside, coco glucoside, lauroyl arginine, sodium cocoyl glycinate and sodium cocoyl isethionate.

9. The shampoo in solid form according to claim 1, wherein the at least one surfactant is sodium lauryl sulfoacetate.

10. The shampoo in solid form according to claim 1, wherein the at least one surfactant is sodium cocoyl glutamate.

11. The shampoo in solid form according to claim 1, wherein the at least one surfactant is sodium cocoyl glycinate.

12. The shampoo in solid form according to any one of claims 1 to 11, wherein the at least one thickening agent is present in an amount of between about 1% and about 30% per total mass of the shampoo.

13. The shampoo in solid form according to any one of claims 1 to 11, wherein the at least one thickening agent is present in an amount of between about 5% and about 20% per total mass of the shampoo.

14. The shampoo in solid form according to any one of claims 1 to 13, wherein the at least one thickening agent is selected from: dehydrated xanthan gum, xanthan gum, cellulose gum, sodium alginate and carrageenan.

15. The shampoo in solid form according to any one of claims 1 to 13, wherein the at least one thickening agent is xanthan gum.

16. The shampoo in solid form according to any one of claims 1 to 13, wherein the at least one thickening agent is carrageenan.

17. The shampoo in solid form according to any one of claims 1 to 16, wherein the at least one conditioning agent is present in an amount of between about 0.1% and about 10% per total mass of the shampoo.

18. The shampoo in solid form according to any one of claims 1 to 17, wherein the at least one conditioning agent is selected from: sarcosine, protein hydrolysates, plant powder, starches, vegetable oils and vegetable milks.

19. The shampoo in solid form according to any one of claims 1 to 17, wherein the at least one conditioning agent is rice starch.

20. The shampoo in solid form according to any one of claims 1 to 19, wherein the at least one preservation agent is present in an amount of between about 0.1% and about 10% per total mass of the shampoo.

21. The shampoo in solid form according to any one of claims 1 to 19, wherein the at least one preservation agent is present in an amount of between about 0.1% and about 5% per total mass of the shampoo.

22. The shampoo in solid form according to any one of claims 1 to 21, wherein the at least one preservation agent is selected from: grapefruit seed extract, gluconolactone, sodium benzoate, naticide, leucidal and potassium sorbate.

23. The shampoo in solid form according to any one of claims 1 to 21, wherein the at least one preservation agent is gluconolactone.

24. The shampoo in solid form according to any one of claims 1 to 21, wherein the at least one preservation agent is sodium benzoate.

25. The shampoo in solid form according to any one of claims 1 to 24, comprising at least one additive.

26. The shampoo in solid form according to claim 25, wherein the at least one preservation agent is present in an amount of between about 0.01% and about 7% per total mass of shampoo.

27. The shampoo in solid form according to claim 25 or 26, wherein at least one additive is a chelating agent.

28. The shampoo in solid form according to claim 27 wherein the chelating agent is tetrasodium glutamate diacetate, sodium gluconate or sodium citrate.

29. The shampoo in solid form according to claim 25 or 26, wherein the at least one additive is a pH adjuster.

30. The shampoo in solid form according to claim 29, wherein the pH adjuster is selected from: citric acid, sodium sesquicarbonate and cream of tartar.

31. The shampoo in solid form according to claim 23, wherein the at least one of the additives includes a fragrance derived from natural sources: where the fragrance derived from natural sources is one of the following: aromatic oils, essential oils or extract of supercritical CO₂.

32. The shampoo in solid form according to claim 25 or 26, wherein the at least one additive is a coloring agent.

33. The shampoo in solid form according to claim 32, wherein the coloring agent is a coloring agent in natural powder form, a liquid coloring or a vegetable extract.

34. The shampoo in solid form according to claim 32 or 33, wherein the at least one additive is an absorbing agent.

35. The shampoo in solid form according to claim 34, where the absorbing agent is selected from: cyclodextrin, tapioca starch, cornstarch and maltodextrin.

36. The shampoo in solid form according to any one of claims 1 to 32, where the aqueous solvent is water.

37. The shampoo in solid form according to any one of claims 11 to 32, further comprising a dissolving agent.

38. The shampoo in solid form according to claim 37, wherein the dissolving agent is present in an amount of between about 10% and about 60% per total mass of the shampoo.

39. The shampoo in solid form according to any one of claims 1 to 38, wherein the solid form is a powder.

40. The shampoo in solid form according to any one of claims 1 to 38, wherein the solid form is a tablet.

41. A method for reconstituting the shampoo in solid form according to any one of claims 1 to 39, the method comprising:
solubilizing the shampoo in solid form in a quantity of aqueous solvent in order to create a solubilized shampoo; and
shaking the solubilized shampoo.
